(19) United States
(12) Patent Application Publication
(10) Pub. No.: US 2004/0053899 A1
(43) Pub. Date: Mar. 18, 2004

(54) Method of preparing a pharmaceutical composition

(76) Inventors: Richard Edward Thomas, New South Wales (AU); Jeffrey Golder, New South Wales (AU)

Correspondence Address: FISH & RICHARDSON PC, 225 FRANKLIN ST, BOSTON, MA, 02110, US

(21) Appl. No.: 10/343,666

(22) PCT Filed: Aug. 2, 2001
(86) PCT No.: PCT/AU01/00942

(30) Foreign Application Priority Data
Aug. 3, 2000 (AU) PQ 9167

(51) Int. Cl. A61K 31/555; A61K 31/28; A61K 31/573

(52) U.S. Cl. 514/171; 514/184; 514/495

(57) ABSTRACT

The present invention relates to a method for the large scale preparation of a viscous pharmaceutical or veterinary composition comprising a heat-sensitive component and, in particular, to a method of preparing an ointment.

### TECHNICAL FIELD

The present invention relates to a method for the large scale preparation of a viscous pharmaceutical or veterinary composition comprising a heat-sensitive component and, in particular, to a method of preparing an ointment.

### BACKGROUND

Traditionally, the preparation of viscous pharmaceutical or veterinary compositions, in particular ointments, on a commercial scale involves heating a suitable ointment base, e.g. paraffin, either prior to, or during, mixing of an active agent with the ointment base to obtain a an homogenous mixture.

Use of this method in the large scale manufacture of a pharmaceutical composition containing an organic gold compound resulted in severe impairment of the therapeutic is properties of the compound. This problem would not have been recognised in the past since-organic gold compounds have generally been prepared as injections, or, in the case of auranofin, as tablets. In ancient and traditional medicine, gold in the form of gold leaf, has been applied to the skin. The traditional method of preparing viscous pharmaceutical and veterinary compositions is therefore not suitable for the large scale production of at least certain compositions comprising organic gold compounds and is likely therefore to be unsuitable for the large scale manufacture of many other compositions containing compounds that have, as yet, not been manufactured as viscous products.

It would therefore be advantageous to provide a method of preparing a pharmaceutical or veterinary composition, particularly a composition comprising an organic gold compound, in which the chemical and/or therapeutic properties of compounds that would otherwise be lost or impaired, may be retained.

It is an object of the present invention to ameliorate or overcome one or more of the deficiencies of the prior art or to provide a useful alternative.

### SUMMARY OF THE INVENTION

In the conventional method for preparing viscous pharmaceutical and veterinary compositions, heat is primarily applied to reduce the viscosity of the ointment base thus enabling easier mixing and packaging.

It has been found that heat applied in the manufacture of a composition containing an organic gold compound and a steroid by the conventional process caused loss of therapeutic activity. This was surprising since the organic gold compound in the composition appeared to be stable as shown by high performance liquid chromoatography (HPLC) and nuclear magnetic resonance (NMR) analysis. It has further been surprisingly found that preparation of substantially the same composition without application of heat and as described below, led to retention of the therapeutic activity of the active ingredients. It appears that the crystal structure of the organic gold compound may have been affected by the heat thus leading to reduced therapeutic efficacy. Clearly, the identification of this problem and the solution described below will be useful in the preparation of many other compositions in which therapeutic efficacy of the active compound is reduced due to the application of heat during large scale manufacture.

According to a first aspect, the invention provides a method for the large scale preparation of a viscous pharmaceutical or veterinary composition comprising an organic gold compound, wherein the method comprises mixing the organic gold compound with a topically acceptable carrier, excipient, solvent or adjuvant, at a temperature and pressure that allows retention of at least some of the therapeutic properties of the compound.

Preferably, the composition further comprises a steroid, more preferably the steroid is a glucocorticosteroid and most preferably the glucocorticosteroid is betamethasone dipropionate.

Preferably, the organic gold compound is auranofin and the steroid is betamethasone dipropionate.

Preferably, the temperature is lower than 60° C., more preferably it is lower than 37° C. and most preferably it is between 20 and 25° C. It will be clear to the skilled addressee that an appropriate temperature and pressure at which to carry out mixing can be determined by simple experimentation.

Preferably mixing is carried out in a vacuum. As air may become trapped in the composition during mixing, this may increase the final volume of the composition and/or lead to oxidation of compounds which are sensitive to oxygen. It will be understood that the application of a vacuum will be particularly useful when either or both of these issues needs to be addressed.

Preferably, the composition is in the form of an ointment.

Preferably, the composition comprises 0.02 to 2% (w/w) organic gold compound, more preferably it comprises 0.1 to 0.5% (w/w) organic gold compound and most preferably it comprises 0.2% (w/w) organic gold compound.

Preferably, the composition comprises 0.001 to 1.0% (w/w) steroid, more preferably it comprises 0.005 to 0.05% (w/w) steroid and most preferably the composition comprises about 0.01% (w/w) steroid. When the steroid is betamethasone dipropionate it is preferably present at about 0.01% and when the steroid is a steroid other than betamethasone dipropionate, it is preferable that an equipotent percentage to that of about 0.01% betamethasone dipropionate is present.

In a preferred embodiment, the composition comprises about 0.2% (w/w) organic gold compound and about 0.01% (w/w) steroid.

Preferably, the composition further comprises a hydrocarbon. Preferably, the hydrocarbon is paraffin. Preferably the hydrocarbon is a mixture of hard, soft and/or liquid paraffin. Preferably, the composition comprises 40 to 95% (w/w) soft paraffin, more preferably it comprises 60 to 90% (w/w) soft paraffin and most preferably it comprises about 80% (w/w) soft paraffin. Preferably the soft paraffin is yellow soft paraffin. It will be clear to the skilled addressee that under certain circumstances it may be useful to use white soft paraffin. It will also be clear to the skilled addressee that it may be desirable to use hard paraffin in some circumstances e.g. when the composition is to be used in regions of high temperature, such as the tropics.

Preferably, the composition comprises 2 to 40% liquid paraffin (w/w), more preferably it comprises 10 to 30% (w/w) liquid paraffin and most preferably it comprises about 20% (w/w) liquid paraffin.

Preferably, the composition comprises about 0.2% (w/w) auranofin; 0.01% (w/w) betamethasone dipropionate; about 80% (w/w) yellow soft paraffin; and about 20% (w/w) liquid paraffin.

According to a second aspect, the invention provides a method for large scale preparation of a viscous pharmaceutical or veterinary composition comprising mixing an organic gold-containing compound and a pharmaceutically acceptable carrier, excipient, solvent or adjuvant, at below 60° C.

Preferably, the composition further comprises a steroid and more preferably the steroid is a glucocorticosteroid.

Preferably, the organic gold compound is auranofin and the glucocorticosteroid is the betamethasone dipropionate.

Preferably, the temperature is lower than 37° C. and most preferably it is between 20 and 25° C.

Preferably mixing is carried out in a vacuum.

According to a third aspect, the present invention provides a viscous pharmaceutical or veterinary composition when prepared according to the method of the first or second aspect.

According to a fourth aspect, there is provided a mixing apparatus when used for the preparation of a composition according to the first or second aspect, in which one or more components are heat-sensitive, comprising:

a vessel;

a lid sealingly engagable with the vessel;

a spindle projecting through and sealingly engagable with the lid;

impellers attached to the spindle wherein, when the apparatus is in use, the impellers are capable of mixing the ingredients such that a homogeneous composition is produced.

Preferably the vessel is vacuum rated and further comprises an air outlet connectable to a vacuum source.

Preferably, the apparatus comprises two impellers.

Preferably, the spindle is sealed to the lid by a mechanical seal. Preferably an O-ring and toggle clamp form a seal between the lid and vessel when the vessel is in use.

According to a fifth aspect, the present invention provides a method of treatment comprising administration of a composition according to the third aspect to a subject in need thereof.

Preferably, the treatment is treatment of a dermatological disorder. Most preferably the dermatological disorder is psoriasis.

According to a sixth aspect, the present invention provides a method of preparing a viscous pharmaceutical or veterinary composition comprising a heat-sensitive compound, wherein the method comprises mixing the heat-sensitive compound with a topically acceptable carrier, excipient, solvent or adjuvant, in an apparatus comprising:

a vessel;

a lid sealingly engagable with the vessel;

a spindle projecting through and sealingly engagable with the lid;

impellers attached to the spindle such that when mixing, the impellers are capable of producing a homogenous composition;

wherein the method is performed at a temperature and pressure that allows retention of at least some of the therapeutic properties of the compound.

In the context of the present invention, the term “viscous pharmaceutical or veterinary composition” should be construed in the sense of a composition which, according to current common manufacturing processes, requires the application of heat in order to allow homogenous mixing and/or dispensing.

In the context of the present invention, the term “paraffin” should be construed according to its known meaning in the field of the invention and includes within its meaning, but is not limited to, Hard Paraffin BP (known in the United States as Paraffin or paraffin wax); Yellow or White Soft Paraffin BP (known in the United States as Liquid Petrolatum or paraffin jelly); and Liquid Paraffin BP (known in the United States as Liquid Petrolatum or mineral oil).

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”.

### BRIEF DESCRIPTION OF THE DRAWINGS

FIG. 1 is a sectional view of a vessel that may be used in the method of the invention or to produce the compositions of the invention.

### DESCRIPTION OF INVENTION

A preferred embodiment of the invention will now be described, by way of example only, and with reference to the accompanying drawing:

### EXAMPLE 1

### Method A (Heat Treatment)

A conventional hot-melt procedure for large scale production of a viscous pharmaceutical comprising the organic gold compound auranofin and the glucocorticosteroid betamethasone dipropionate was carried out as follows:

1. 150 kg Yellow Soft Paraffin BP was placed in a 250 L manufacturing kettle, heated to 60° C., maintained at this temperature and mixed using an Anchor mixer at 10 rpm when the mass became molten.

2. 5 kg Molten Yellow Soft Paraffin BP from step 1 was added to a 25 L stainless steel vessel and homogenised with 440 kg auranofin and 22 kg betamethasone dipropionate for 6-8 minutes.

3. 5 kg Molten Yellow Soft Paraffin BP from step 1 was placed in a 50 L stainless steel vessel.

4. Material from step 2 was placed in the 250 L manufacturing kettle. The 25 L stainless steel vessel was rinsed with the material from step 3 and the rinsings added to the 250 L manufacturing kettle.

5. The mixture of step 4 was homogenised for 20 min.

6. 69.5 kg Yellow Soft Paraffin was added to a 100 L phase kettle and heated to 60° C. The mass was stirred when it became molten. Heating and stirring was continued until the mass was completely melted.

7. Melted material from step 6 was added to the 250 L manufacturing kettle.

8. The mixture of step 7 was homogenised for 20 min.

9. The auranofin/betamethasone dipropionate mixture of step 8 was collected and is the mixture referred to as the product of method A (heat treated).

The ointment produced by this method contained 0.203% w/w auranofin and 0.010% w/w betamethasone dipropionate.

### EXAMPLE 2

### Results of a Randomized Clinical Trial of Compositions Produced by Method A (Heat Treatment) Described Above.

This study was a Phase IIb trial performed in compliance with Australian Good Clinical Practices (GCP) guidelines. The aim was to determine whether a topical composition containing auranofin (0.2%) and betamethasone dipropionate (0.01%) produced by Method A (heat treatment) above provides a clinically relevant improvement in stable chronic plaque-type psoriasis. The study was a single centre, double blind, randomised, parallel study with all lesions treated except those lesions in the flexures or on the face and scalp.

One hundred and forty-four patients participated in the trial. Subjects were healthy males or females, aged 18-70 years with chronic (≧12 months), plaque-type psoriatic lesions to no more than 30% of their body surface area. Each of the patients were treated over an eight-week period with a two week washout before and after the treatment period.

Treatments

The trial compared two products designated ‘test’ and ‘active comparator’.

The test product contained auranofin 0.2% w/w and betamethasone dipropionate 0.01% in Yellow Soft Paraffin BP.

The active comparator product contained betamethasone dipropionate 0.05% in Yellow Soft Paraffin BP. Betamethasone dipropionate in this concentration is widely used in topical products for the treatment of psoriasis and is generally regarded by dermatologists as the preferred topical steroid for treating this condition. The comparator product is thus an appropriate ‘gold standard’ against which to compare a new topical treatment for the treatment of psoriasis.

Both products were prepared by Method A (heat treatment), were packaged in identical containers and could not be distinguished by visual inspection of either the container or its contents.

Randomization

Each product was given a number that corresponded to the patient's randomized number. Neither the patient nor any of the evaluators knew which product was test and which was comparator. Patients were randomized and given test or comparator product according to the randomization protocol.

Efficacy and Safety

Efficacy was evaluated by one objective measure, the Psoriasis Area Severity Index (PASI) scale, and two subjective measures, namely, the Patient Global Assessment of Product Efficacy (PGA) and the Psoriasis Disability Index (PDI) derived from the patient self-completion quality of life questionnaires. All three measures are described in the literature and are widely used and respected by psoriasis investigators. Published studies have validated these tests as suitable for evaluating treatment of psoriasis.

Results

During the course of the trial, mean PASI scores fell by the following amounts.

| | Percent fall in PAST score |
|---|---|
| | over trial period (mean ± SEM) |
| Patients treated with Active Comparator (n = 73) | 49.56 ± 2.69% |
| Patients treated with Test Product (n = 71) | 23.01 ± 3.74 |

The difference between the two results was statistically significant (p=0.0001).

Thus, the test product was clearly less effective than the comparator product. This does not rule out the possibility of a significant effect from the test product but the product is clearly less effective than the comparator product (i.e. the ‘gold standard’).

The secondary (subjective) efficacy variables (PGA and PDI) supported the primary (objective) efficacy outcomes (PASI results).

In previous open label studies using a test batch of the composition prepared on a small scale at ambient temperature by a pharmacist (in accordance with a method described in PCT AU 89/00118), high levels of efficacy were recorded using qualitative measures. Therefore, it was surprising that the large scale composition made by the conventional methods (heat treatment) showed little or no efficacy in the randomized controlled study.

Safety

Both test and control compositions were associated with a similar level of adverse events during the course of treatment. Analysis of these events indicated that none was related to the use of either composition.

Blood analyses showed that detectable levels of gold were not present during or at the end of the treatment period.

### EXAMPLE 3

### Method B (No Heat Treatment)

The following describes a method of producing an ointment comprising auranofin and betamethasone dipropionate, in which no heat is applied:

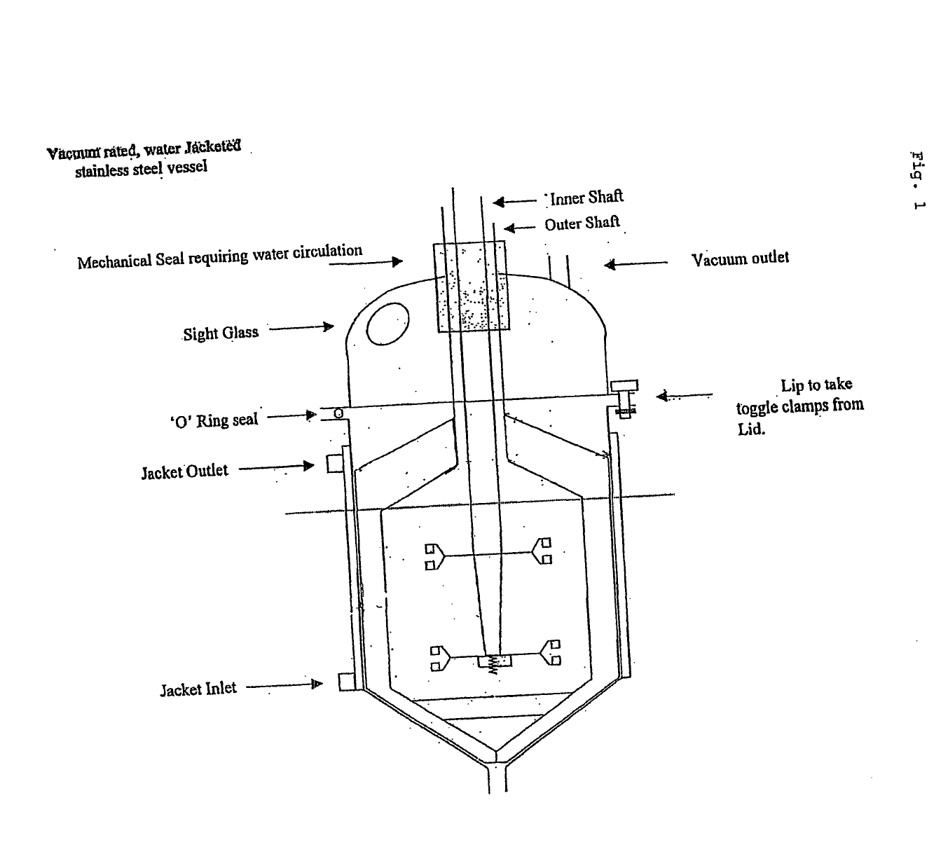

The method uses a mixing blade and does not require heat to melt the Yellow Soft Paraffin BP prior to, or during, mixing. The mixing blade used in this example is shown in FIG. 1. Mixing was carried out under vacuum.

The ointment was prepared in the following way:

6 g of auranofin and 0.3 g of betamethasone dipropionate were mixed by hand with 10 g of Yellow Soft Paraffin BP using a spatula on a glass mixing plate.

To the mixing equipment shown in FIG. 1, 2384 g of Yellow Soft Paraffin BP, 598 mL of liquid paraffin and the 16.3 g of the mixture above was added.

The mixing vessel was sealed with a perspex lid and the remaining air inside the mixing vessel was evacuated using an electric pump.

The contents of the vessel were then mixed at room temperature (20-25° C.) for 5-10 min at up to 2000 rpm.

The ointment was then removed using large spatulas and placed into 100 g ointment containers for use.

Six containers were selected at random and auranofin concentrations were measured from three layers (top middle and bottom) of each container.

Analysis of the six containers showed the product to have a mean auranofin concentration of 0.183% with a 95% confidence interval of ±0.037.

### EXAMPLE 4

### Study of Possible Cause of Loss Efficacy

Because of the very promising results obtained using cold-prepared ointment prepared by a pharmacist, and the negative results obtained using the large scale preparation, it was hypothesized that the auranofin had undergone chemical decomposition during the heating process employed in the large scale method. This was investigated exhaustively using HPLC, TLC and proton NMR to compare the compositions prepared in accordance with Method A (heat treatment—Example 1 above) and compositions prepared in accordance with Method B (no heat treatment—Example 3 above). These studies were carried out on the samples used in the trial and in a series of model settings. NMR spectroscopy was conducted by qualified staff of the School of Chemistry, University of New South Wales. All other analytical studies were conducted qualified staff of Insearch Limited, University of Technology, Sydney. The results showed conclusively that no chemical decomposition had occurred. It should be emphasised that the results of the HPLC analysis would normally be accepted by regulatory authorities as an indication that the compositions (prepared by the two different methods) would have the same physical/chemical properties.

In view of the surprising result that no chemical decomposition had occurred during the heating process, it was hypothesized that changes had occurred to particle size, thus changing the release characteristics of the auranofin from the ointment base. Particle size analysis of solids in an ointment containing auranofin (0.2%) and betamethasone dipropionate (0.01%) prepared without heat and with heat were conducted by the Electron Microscopy Unit of the University of Sydney. The results of this study are shown in Table 1.

Table 1

Particles in the composition were classified according to four categories:

| | |
|---|---|
| Size: | S, M or L (small, medium or large) |
| Surface: | R or S (rough or smooth) |
| Small particles like small dots: | G if present, dash if absent |
| Shape: | R or S (round or sharp) |

Hence, for example, the term SSGR indicates that a particle was small, smooth, dots included and round, whereas LR-S indicates that a particle was large, rough no particles included and had sharp sides.

| | Heat-Treated Composition | | Cold Mixed Composition | |
|---|---|---|---|---|
| Classification | Number | % | Number | % |

Small Particle Size

| | | | | |
|---|---|---|---|---|
| SSGR | 36 | 26 | 2 | 1 |
| SS-R | 10 | 7 | 0 | 0 |
| SS-S | 4 | 3 | 14 | 10 |
| SR-S | 1 | 1 | 0 | 0 |
| SR-R | 0 | 0 | 0 | 0 |
| SUBTOTAL | 51 | 37 | 16 | 11 |

Medium Particle Size

| | | | | |
|---|---|---|---|---|
| MSGR | 5 | 4 | 3 | 2 |
| MS-R | 3 | 2 | 0 | 0 |
| MS-S | 45 | 33 | 51 | 35 |
| MR-R | 0 | 0 | 4 | 3 |
| MR-S | 12 | 9 | 34 | 23 |
| SUBTOTAL | 65 | 47 | 92 | 63 |

Large Particle Size

| | | | | |
|---|---|---|---|---|
| LSGR | 0 | 0 | 0 | 0 |
| LS-R | 3 | 2 | 0 | 0 |
| LS-S | 8 | 6 | 11 | 8 |
| LR-S | 11 | 8 | 26 | 18 |
| SUBTOTAL | 22 | 16 | 37 | 26 |
| TOTAL | 138 | 100 | 145 | 100 |

These studies showed that heating caused a reduction in the number and size of visible particles. Assuming that the particles observed were spheres, the results showed a 75% reduction in the volume of material held in visible particles (Table 2).

TABLE 2

| | NOT HEATED | | | | HEATED | | | |
|---|---|---|---|---|---|---|---|---|
| | Number | | | | Number | | | |
| | of | Average | Avemge | Total | of | Average | Average | Total |
| | particles | diameter | volume | volume | particles | diameter | volume | volume |
| Small | 16 | 7 | 180 | 2,874 | 51 | 3 | 14 | 721 |
| Intermediate | 92 | 16 | 2,145 | 197,309 | 65 | 11 | 697 | 45,299 |
| Large | 37 | 28 | 11,494 | 425,279 | 22 | 21 | 4849 | 106,679 |
| All particles | 145 | 18.1 | 4313 | 625,462 | 138 | 9.6 | 1101 | 151,978 |

Note:
The ratio of auranofin to betamethasone in the ointment is 20:1. This means that some of the particles observed could be the steroid.

Table 2 shows that a reduction in average diameter of about 50%, corresponds to a total volume reduction of about 75%. This assumption is based on the particles being perfect spheres. This of course is not the case. There is no simple way of calculating the volume of irregular shaped powder particles, particularly if these are highly etched, as was the case with the heated ointment. However, one very important point can be made, namely, the more irregular tile particle shape, the faster will it leave the base and enter the skin. Microscopy showed that crystals from the heated ointment were far more irregular than those in the cold-prepared product.

Implications of the Particle Size Studies

The particle size studies have implications that are counter-intuitive. Those skilled in the art of making dermatological products spend much effort in formulating products that promote rapid transfer from the product vehicle to the skin. For active ingredients in particulate form, the speed of release is inversely proportional to the size of the particles. The data in Table 2 indicate that some of the active ingredient had dissolved in the base (or was in ultra-fine suspension) and the remaining particles were smaller and had highly etched surfaces. This means that heating changed the physical properties of the active ingredients in such a way that faster and more complete delivery of the actives into the skin would normally be expected. Accordingly, a more effective product would be expected. Surprisingly, as shown in Example 5, this was not the case.

### EXAMPLE 5

### Results of a Randomized Clinical Trial of Ointments Produced by Method B (No Heat Treatment)

This study was a Phase IIb trial performed in compliance with Australian Good Clinical Practices (GCP) guidelines. The aim was to determine whether a topical product containing auranofin (0.2%) and betamethasone dipropionate (0.01%) produces a clinically relevant improvement in stable chronic plaque-type psoriasis. The study was a single centre, double-blind, randomised, parallel study in which one lesion on each side of the patient was treated with test product and one with active comparator. Thus, each patient acted as his own control.

Twenty patients participated in the trial. Subjects were healthy males or females, aged 18-70 years with chronic (≧12 months), plaque-type psoriatic lesions to no more than 30% of their body surface area. Each of the patients were treated over an eight-week period with a two week washout period before and after the treatment period.

Treatments

The trial compared two products designated ‘test’ and ‘active comparator’. The test product contained auranofin 0.2% (w/w) and betamethasone dipropionate 0.01% (w/w) in Yellow Soft Paraffin BP. The active comparator product contained betamethasone dipropionate 0.05% (w/w) in Yellow Soft Paraffin BP.

Both products were prepared at room temperature using Method B (no heat treatment), were packaged in identical containers and could not be distinguished by visual inspection of either the container or its contents. Products were labelled either ‘left’ or ‘right’

Randomization

Products and patients were randomized so that each patient received one set of containers marked ‘left’ and one set marked ‘right’. Each set contained either test product or active comparator. In some cases the test was in the ‘right’ container and sometimes in the ‘left’. Neither patients nor investigators were aware of which was which.

Efficacy and Safety

This study differed from that in Example 2 in that the total body lesions were not being treated. Therefore the PASI scale, as described in the literature, is not applicable. Instead, a similar scale that is directly comparable with PASI was used. This was a composite scale using total lesion area (TLA) combined with a Total Severity Score (TSS) that sums scores for redness, scaling, and thickness. Together, these two measures are equivalent to PASI. These measures were supplemented by two subjective measures, namely, the Patient Global Assessment of Product Efficacy (PGA) and the Psoriasis Disability Index (PDI) derived from the patient self-completion quality of life questionnaires. All these measures are described in the literature and are widely used and respected by psoriasis investigators. Published studies have validated these tests as suitable for evaluating treatment of psoriasis.

Results

During the course of the trial, mean TLA and TSI scores fell as follows.

| | Percent fall over trial period | |
|---|---|---|
| | (mean ± SEM) | |
| | TLA | TSI |
| Patients treated with Active Comparator (n = 20) | 26.7 ± 1.89 | 57.7 ± 39.6 |
| Patients treated with Test Product (n = 20) | 23.6 ± 1.95 | 55.3 ± 46.53 |

The difference between the two sets of results was not statistically significant (p=0.211 for TLA and 0.442 for TSI).

The secondary (subjective) efficacy variables (PGA and PDI) supported the primary (objective) efficacy outcomes (TLA and TSI). Safety

Both test and control products were associated with a similar level of adverse events during the course of treatment. Analysis of these events indicated that none was related to the use of either product.

Blood analyses showed that detectable levels of gold were not present during or at the end of the treatment period.

There were no cases of contact dermatitis.

Thus, in the Examples provided above it is clear that an auranofin-containing composition, when prepared by a method which does not involve heat treatment, has superior therapeutic properties compared to a composition prepared by the traditional method involving heat treatment, even when standard chemical indicators are the same for both compositions.

Although the invention has been described with reference to specific examples, it will be appreciated by those skilled in the art that the invention may be embodied in many other forms.

The claims defining the invention are as follows:

1. A method for the large scale preparation of a viscous pharmaceutical or veterinary composition comprising an organic gold compound, wherein the method comprises mixing the organic gold compound with a topically acceptable carrier, excipient, solvent or adjuvant, at a temperature and pressure that allows retention of at least some of the therapeutic properties of the compound.

2. A method according to claim 1 wherein the composition further comprises a steroid.

3. A method according to claim 2 wherein the steroid is a glucocorticosteroid.

4. A method according to claim 3 wherein the glucocorticosteroid is betamethasone dipropionate.

5. A method according to any one of claims 1 to 4 wherein the organic gold compound is auranofin.

6. A method according to claim 1 wherein the gold compound is auranofin and the steroid is betamethasone dipropionate.

7. A method according to any one of claims 1 to 6 wherein the temperature is lower than 60° C.

8. A method according to claim 7 wherein the temperature is lower than 37° C.

9. A method according to claim 8 wherein the temperature is between 20 and 25° C.

10. A method according to any one of claims 1 to 9 wherein mixing is carried out in a vacuum.

11. A method according to any one of claims 1 to 10 wherein the composition is in the form of an ointment.

12. A method according to any one of claims 1 to 11 wherein the composition comprises 0.02 to 2% (w/w) organic gold compound.

13. A method according to any one of claims 1 to 12 wherein the composition comprises 0.1 to 0.5% (w/w) organic gold compound.

14. A method according to claim 13 wherein the composition comprises 0.2% (w/w) organic gold compound.

15. A method according to any one of claims 1 to 14 wherein the composition comprises 0.001 to 1.0% (w/w) steroid.

16. A method according to claim 15 wherein the composition comprises 0.005 to 0.05% (w/w) steroid.

17. A method according to claim 16 wherein the composition comprises about 0.01% (w/w) steroid.

18. A method according to any one of claims 1 to 11 wherein the composition comprises about 0.2% (w/w) organic gold compound and about 0.01% (w/w) steroid.

19. A method according to any one of claims 1 to 18 wherein the composition further comprises a hydrocarbon.

20. A method according to claim 19 wherein the hydrocarbon is paraffin.

21. A method according to claim 20 wherein the hydrocarbon is a mixture of hard, soft and/or liquid paraffin.

22. A method according to claim 20 or claim 21 wherein the composition comprises 40 to 95% (w/w) soft paraffin.

23. A method according to claim 22 wherein the composition comprises 60 to 90% (w/w) soft paraffin.

24. A method according to claim 23 wherein the composition comprises about 80% (w/w) soft paraffin.

25. A method according to any one of claims 22 to 24 wherein the soft paraffin is yellow soft paraffin.

26. A method according to any one of claims 22 to 24 wherein the soft paraffin is white soft paraffin.

27. A method according to claim 20 or claim 21 wherein the paraffin is hard paraffin.

28. A method according to any one of claims 20 to 27 wherein the composition comprises 2 to 40% (w/w) liquid paraffin.

29. A method according to claim 28 wherein the composition comprises 10 to 30% (w/w) liquid paraffin.

30. A method according to claim 29 wherein the composition comprises about 20% (w/w) liquid paraffin.

31. A method according to any one of claims 1 or 7 to 11, wherein the composition comprises about 0.2% (w/w) auranofin, 0.01% (w/w) betamethasone dipropionate, about 80% (w/w) yellow soft paraffin and about 20% (w/w) liquid paraffin.

32. A method for large scale preparation of a viscous pharmaceutical or veterinary composition comprising mixing an organic gold-containing compound and a pharmaceutically acceptable carrier, excipient, solvent or adjuvant, at below 60° C.

33. A method according to claim 32 wherein the composition further comprises a steroid.

34. A method according to claim 33 wherein the steroid is a glucocorticosteroid.

35. A method according to any one of claim 32 to 34 wherein the organic gold compound is auranofin

36. A method according to claim 32 wherein the organic gold compound is auranofin and the glucocorticosteroid is betamethasone dipropionate.

37. A method according to any one of claims 32 to 36 wherein the temperature is lower than 37° C.

38. A method according to claim 37 wherein the temperature is between 20 and 25° C.

39. A method according to any one of claims 32 to 38 wherein the mixing is carried out in a vacuum.

40. A viscous pharmaceutical or veterinary composition when prepared according to the method of any one of claims 1 to 39.

41. A mixing apparatus when used for the preparation of a composition according to any one of claims 1 to 39 wherein one or more of the components of the composition are heat-sensitive, comprising:
(a) a vessel;
(b) a lid sealingly engagable with the vessel;
(c) a spindle projecting through and sealingly engagable with the lid;
(d) impellers attached to the spindle wherein, when the apparatus is in use, the impellers are capable of mixing the ingredients such that a homogeneous composition is produced.

42. A mixing apparatus according to claim 41 wherein the vessel is vacuum rated and further comprises an air outlet connectable to a vacuum source.

43. A mixing apparatus according to claim 41 or claim 41 wherein the apparatus comprises two impellers.

44. A mixing apparatus according to any one of claims 41 to 43 wherein the spindle is sealed to the lid by a mechanical seal.

45. A mixing apparatus according to any one of claims 41 to 43 wherein an O-ring and toggle clamp form a seal between the lid and vessel when the vessel is in use.

46. A method of treatment comprising administration of a composition according to claim 40 to a subject in need thereof.

47. A method according to claim 46 wherein the treatment is treatment of a dermatological condition.

48. A method according to claim 47 wherein the dermatological condition is psoriasis.

49. A method of preparing a viscous pharmaceutical or veterinary composition comprising a heat-sensitive compound, wherein the method comprises mixing the heat-sensitive compound with a topically acceptable carrier, excipient, solvent or adjuvant, in an apparatus comprising:
(a) a vessel;
(b) a lid sealingly engagable with the vessel;
(c) a spindle projecting through and sealingly engagable with the lid;
(d) impellers attached to the spindle such that when mixing, the impellers are capable of producing a homogenous composition;
wherein the method is performed at a temperature and pressure that allows retention of at least some of the therapeutic properties of the compound.
